(19) United States
(12) Patent Application Publication
(10) Pub. No.: US 2023/0321380 A1
(43) Pub. Date: Oct. 12, 2023

(54) ENDOTRACHEAL TUBE FOR TRACHEAL INTUBATION

(71) Applicant: Juan N. Walterspiel, Menlo Park, CA (US)

(72) Inventor: Juan N. Walterspiel, Menlo Park, CA (US)

(21) Appl. No.: 18/132,684

(22) Filed: Apr. 10, 2023

Related U.S. Application Data
(60) Provisional application No. 63/329,880, filed on Apr. 11, 2022.
(60) Provisional application No. 63/392,325, filed on Jul. 26, 2022.
(60) Provisional application No. 63/396,524, filed on Aug. 9, 2022.

(51) Int. Cl. A61M 16/04; A61M 16/08; A61M 39/20

(52) U.S. Cl. CPC A61M 16/0488; A61M 16/0816; A61M 39/20; A61M 16/0486; A61M 16/0461; A61M 25/0147

(57) ABSTRACT

An endotracheal tube assembly and methods of use are disclosed. The endotracheal tube assembly may include a tube having a distal end and a proximal end, a pull line extending through the tube, and a guide structure configured to guide a proximal portion of the pull line at a location proximal of the tube. The pull line may be configured such that when tension is applied to the pull line extending through the guide structure, the distal end of the endotracheal tube may bend in response to the tension applied to the pull line to form a curvature in the tube.

### CROSS REFERENCE TO RELATED APPLICATIONS

This application claims the benefit of U.S. Provisional Patent Application Ser. No. 63/329,880, filed Apr. 11, 2022 and U.S. Provisional Patent Application Ser. No. 63/392,325, filed Jul. 26, 2022 and U.S. Provisional Patent Application Ser. No. 63/396,524, filed Aug. 9, 2022, the disclosures of which are incorporated herein by reference in their entirety for any and all purposes.

### TECHNICAL FIELD

This disclosure relates generally to tube assemblies, systems, and methods, and particularly to user adjustable tubes assemblies, systems, and methods.

### BACKGROUND

A wide variety of intracorporeal medical devices and systems have been developed for medical use, for example, for intubations. Some of these devices and systems include guidewires, tubes, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This summary of the disclosure is given to aid understanding and each of the various aspects and features of the disclosure may advantageously be used separately in some instances, or in combination with other aspects and features of the disclosure in other instances. While the disclosure is presented in terms of aspects or embodiments, it should be appreciated that individual aspects can be claimed separately or in combination with aspects and features of that embodiment or any other embodiment

An example tube assembly may include a tube having a distal end and a proximal end, a pull line extending through the tube, a guide configured to guide a proximal portion of the pull line at a location proximal of the tube, and wherein the pull line is configured relative to the tube and the guide such that when tension is applied to the pull line, the tube bends in response to the tension applied to the pull line to form a curvature in the tube.

Alternatively or additionally to any of the examples above, the tube assembly may include a connector coupled to the proximal end of the tube.

Alternatively or additionally to any of the examples above, the tube assembly may include a connector coupled to the proximal end of the tube, wherein the connector at least partially defines the guide.

Alternatively or additionally to any of the examples above, the guide comprises a first guide and a second guide.

Alternatively or additionally to any of the examples above, the first guide is located at a first circumferential location along the connector and the second guide is located at a second circumferential location along the connector, the second circumferential location is at a side of the connector opposite a side at which the first circumferential location is positioned.

Alternatively or additionally to any of the examples above, the guide may comprise a first guide located at a first circumferential location along the connector and a second guide located at a second circumferential location along the connector, the second circumferential location is at a side of the connector opposite a side at which the first circumferential location is positioned.

Alternatively or additionally to any of the examples above, the guide may be or may include a channel structure attached to the connector and configured to receive the pull line.

Alternatively or additionally to any of the examples above, the guide may be or may include a channel structure integrated into the connector and configured to receive the pull line.

Alternatively or additionally to any of the examples above, the guide may include a channel defined by an outer periphery of the guide and configured to receive the pull line.

Alternatively or additionally to any of the examples above, the guide may have one or more guiding slits or slots configured to receive the pull line.

Alternatively or additionally to any of the examples above, the guide may comprise a first guide and a second guide.

Alternatively or additionally to any of the examples above, the first guide may be located distal of the second guide.

Alternatively or additionally to any of the examples above, the first guide may be circumferentially offset from the second guide.

Alternatively or additionally to any of the examples above, the guide may comprise a first guide and a second guide, the first guide may be located distal of the second guide.

Alternatively or additionally to any of the examples above, the tube assembly may further include a pull component coupled to a proximal end of the pull line, wherein the pull component and the pull line may be configured such that a tension is applied to the pull line when a user pulls on the pull component.

Alternatively or additionally to any of the examples above, the tube comprises an airway lumen and a pull line lumen.

Alternatively or additionally to any of the examples above, a distal end of the pull line is coupled to the tube at a location within the pull line lumen.

Alternatively or additionally to any of the examples above, the pull line exits the tube at an orifice in communication with a proximal end of the pull line lumen.

Alternatively or additionally to any of the examples above, the guide structure may be configured to guide the pull line in a proximal direction from an orifice in a first side of the tube to a location proximate a second side of the tube.

Alternatively or additionally to any of the examples above, the tube assembly may comprise a connector coupled to the proximal end of the tube, wherein the guide may include a frustoconical ring extending around the connector.

Alternatively or additionally to any of the examples above, the frustoconical ring may include a V-shape slit opening toward the first side of the tube, and the slit may be configured to receive the pull line and when tension is applied to the pull line, the tube bends in response to the tension applied to the pull line to form the curvature in the tube

Alternatively or additionally to any of the examples above, the tube assembly may further include a cap configured to engage the connector, the cap may define the guide.

Alternatively or additionally to any of the examples above, the tube assembly may further include a compression component, and a first end of the compression component may be secured to the cap, a second end of the compression component may be coupled to the pull line, and the pull line may be configured to extend through the guide defined by the cap.

Alternatively or additionally to any of the examples above, the tube assembly may further include a cap defining the guide and a compression component, and the compression component may be secured to the cap, the pull line extends through the guide defined by the cap, and a bend couples the compression component and the pull line such that the compression component extends between a location at which the first portion is secured to the cap and the bend and the pull line extends from the bend through the guide.

In a further example, an endotracheal tube adaptor may include a cap defining a guide and a holder, the cap is configured to couple to a connector of an endotracheal tube, a pull component, and an adjustment member having a first end, a second end, a first portion extending from the first end, and a second portion extending from the second end, and the first end of the adjustment member is coupled to the holder, the second portion of the adjustment member extends through the guide, and the second end of the adjustment member is coupled to the pull component.

Alternatively or additionally to any of the examples above, the adjustment member may include a bend extending between the first portion and the second portion.

Alternatively or additionally to any of the examples above, the guide may be positioned radially outward from the holder.

Alternatively or additionally to any of the examples above, the holder and the guide may leave space to allow a user to observe entry of the adjustment member into the endotracheal tube through the connector and detect air movements and/or exhaled carbon dioxide.

Alternatively or additionally to any of the examples above, the holder may be located at a central axis of the cap.

In a further example, a method may comprise inserting an endotracheal tube into a mouth or nose of a subject, the endotracheal tube including a connector and a tube extending distally from the connector, providing tension on a pull line extending through a guide located proximate the connector to apply a curve to the tube, and advancing the endotracheal tube with the curve over an epiglottis and into a trachea of the subject.

Alternatively or additionally to any of the examples above, the method may further include threading the pull line through the guide.

Alternatively or additionally to any of the examples above, a distal end of the pull line may be coupled to the distal end of the tube.

Alternatively or additionally to any of the examples above, a distal end of the pull line is coupled to a distal end of a compression component coupled to the connector.

The above summary of some embodiments is not intended to describe each disclosed embodiment or every implementation of the present disclosure.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of an illustrative use of a laryngoscope on a subject and a path through the subject for positioning an endotracheal tube;

FIG. 2 depicts a schematic view of a user engaging an illustrative endotracheal tube assembly;

FIG. 3 depicts a schematic view of a cross-section through an endotracheal tube of the illustrative endotracheal tube assembly in FIG. 2;

FIG. 4 depicts a schematic side view of a portion of an illustrative endotracheal tube assembly;

FIG. 5 depicts a schematic distal end view of an illustrative endotracheal tube assembly with a portion of a tube thereof removed;

FIG. 6 depicts a schematic proximal end view of a portion of an illustrative endotracheal tube assembly;

FIG. 7 depicts a schematic perspective view of an illustrative endotracheal tube connector;

FIG. 8 depicts a schematic view of a portion of an illustrative endotracheal tube assembly;

FIG. 9 depicts a schematic view of an illustrative endotracheal tube assembly;

FIG. 10 depicts a schematic view of the illustrative endotracheal tube assembly depicted in FIG. 9;

FIG. 11 depicts a schematic end view of an illustrative guide of the endotracheal tube assembly depicted in FIG. 9;

FIG. 12 depicts a schematic side view of a portion of an illustrative endotracheal tube assembly;

FIG. 13 depicts a schematic side view of an illustrative endotracheal tube assembly;

FIGS. 14A and 14B depict a schematic side view of the endotracheal tube assembly depicted in FIG. 13 in a resting position and a tensioned position, respectively;

FIG. 15 depicts a schematic side view of a portion of the endotracheal tube assembly depicted in FIG. 13;

FIG. 16 depicts a schematic side view of an illustrative endotracheal tube adaptor of the endotracheal tube assembly depicted in FIG. 13;

FIG. 17 depicts a schematic end view of the illustrative endotracheal tube assembly depicted in FIG. 13;

FIG. 18 depicts a schematic end perspective view of an illustrative cap;

and

FIG. 19 depicts a schematic diagram of an illustrative method of intubating a subject using an endotracheal tube assembly.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical assembly that may be used in tracheal intubation procedures and/or other suitable procedures. However, it should be noted that reference to this particular procedure is provided only for convenience and is not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Various elements described herein may be arranged in various combinations and configurations without departing from the scope of the disclosure. The detailed description illustrates example embodiments of the disclosure.

Tracheal intubation is a procedure that may be performed on subjects of all ages, including preterm infants in delivery rooms and neonatal intensive care units. See e.g., Sweet D G, Carnielli V, Greisen G, Hallman M, Ozek E, to Pas A, et al. European consensus guidelines on the management of respiratory distress syndrome-2019 update, Neonatology 2019; 115:432-50, which is hereby incorporated by reference in its entirety for all purposes. Although often essential for providing lifesaving care, tracheal intubation is a technically challenging procedure and is associated with many potential adverse events, such as cardiorespiratory decompensation and increased risk of morbidity and mortality. See e.g., Sawyer T, Foglia E, Hatch L D, Moussa A, Ades A, Johnston L, et al. Improving neonatal intubation safety: a journey of a thousand miles, J Neonatal-Perinat Med. 2017; 10:125-31, which is hereby incorporated by reference in its entirety for all purposes.

One concern associated with tracheal intubation is the risk of intraventricular hemorrhage in preterm infants, especially among infants of very low gestational age. See e.g., Gleiβner M, Jorch G, Avenarius S. Risk factors for intraventricular hemorrhage in a birth cohort of 3721 premature infants, J Perinat Med. 2000; 28, Available from: https://www.degruyter.com/doi/10.1515/JPM.2000.013, which is hereby incorporated by reference in its entirety for all purposes. Preterm infants are inherently at risk of intraventricular bleed from the structural immaturity (i.e., fragile germinal matrix) and physiologic vulnerability (i.e., impaired autoregulation) noted in the first few days and weeks of their life. See e.g., Ballabh P. Intraventricular hemorrhage in premature infants: mechanism of dis-ease, Pediatr Res. 2010; 67:1-8, which is hereby incorporated by reference in its entirety for all purposes. Hemodynamic instability, changes in cerebral blood flow, and changes in blood pressure associated with tracheal intubations may exacerbate the risk of intraventricular hemorrhage (IVH) and impact the morbidity, mortality, and long-term neurodevelopmental outcomes of preterm infants. See e.g., Bassan H. Intracranial hemorrhage in the preterm infant: understanding it, preventing it, Clin Perinatol. 2009; 36:737-62, which is hereby incorporated by references in its entirety for all purposes. The risk of IVH may be further increased with multiple attempts at tracheal intubation, and multiple attempts are frequent in neonatal intubations. See e.g., Sawyer et al. Foglia et al. reported that only 49% of tracheal intubations in the neonatal intensive care unit and 46% of tracheal intubations in the delivery room were successful on the first attempt. See e.g., Foglia E E, Ades A, Sawyer T, Glass K M, Singh N, Jung P, et al. Neonatal intubation practice and outcomes: an international registry study, Pediatrics 2019; 143: e20180902, which is hereby incorporated by reference in its entirety for all purposes. Applicant has found that tube assemblies and/or tools that facilitate reducing the number of attempts needed to place an endotracheal tube and reduce time needed for such placement may facilitate reducing the risk of IVH and improving the life and/or life expectancy of prenatal infants and other subjects.

Intubating premature Infants with a finger operated endotracheal tube pull line and has been shown to be faster and safer than conventional intubation, Poster #482, Pediatric Academic Societies Conference (PAS) 2022. Denver, Co. Apr. 23, 2022, which is hereby incorporated by reference in its entirety for all purposes. An example catheter for introduction into the trachea of a subject, where the catheter includes a flexible line that may be pulled to apply tension or traction thereto to shape the catheter is disclosed in US2020/0155807A1, filed on Jun. 6,2019, with a title of SMALL DIAMETER CATHETER FOR INTRODUCTION INTO THE TRACHEA AND OTHER ORIFICES, AS WELL AS INTO PASSAGES THAT ARE DIFFICULT TO INTUBATE OR ACCESS. Further, an example endotracheal tube assembly with an endotracheal tube and a traction string, where the endotracheal tube has an airflow lumen and a traction string conduit comprising a traction string, is disclosed in WO2017/031413A1, filed on Aug. 19, 2016, with a title of ENDOTRACHEAL TUBE WITH TRACTION STRING TO FACILITATE INTUBATION, which is hereby incorporated by reference in its entirety for all purposes. However, as the traction string or flexible lines disclosed in known configurations of endotracheal tube assemblies exit a conduit at a side of the endotracheal tube and directly couple to a pull component (e.g., a pull ring or other suitable pull component) located at the side of the endotracheal tube, applying tension or traction to the pull ring and/or the traction string may cause the endotracheal tube to rotate in unintended manners, which may increase the difficulties of intubating a subject with the endotracheal tube.

Endotracheal tube assemblies with a pull line coupled with a pull ring at a proximal end of the pull line and a corresponding pull line routing connector (e.g., a guide structure) that prevents and/or mitigates rotation of the endotracheal tube during intubation are disclosed herein. In one example, an endotracheal tube assembly may include single pull line integrated within an endotracheal tube and a proximal end connected to a pull-ring, where an endotracheal tube connector defines a guide structure. In another example, an endotracheal tube assembly may include an adaptor having an adaptor connector (e.g., a cap and/or other suitable connector) and an adjustment member where the adaptor connector is configured to engage with an endotracheal tube connector and the adjustment member includes a first portion or a pull line configured to extend into an endotracheal tube and a second portion or pull line extending through a guide structure of the adaptor connector with a proximal end connected to a pull ring. In operation of the endotracheal tube assemblies during an intubation, a tube of the endotracheal tube remains open throughout intubation while allowing a user to move the tube as it is inserted into a trachea of a subject by applying force to the pull ring. Once the endotracheal tube is properly placed within a subject and the subject is intubated, the force may be removed from the pull ring and the pull ring may be removed (e.g., cut off or otherwise removed) from the endotracheal tube assembly.

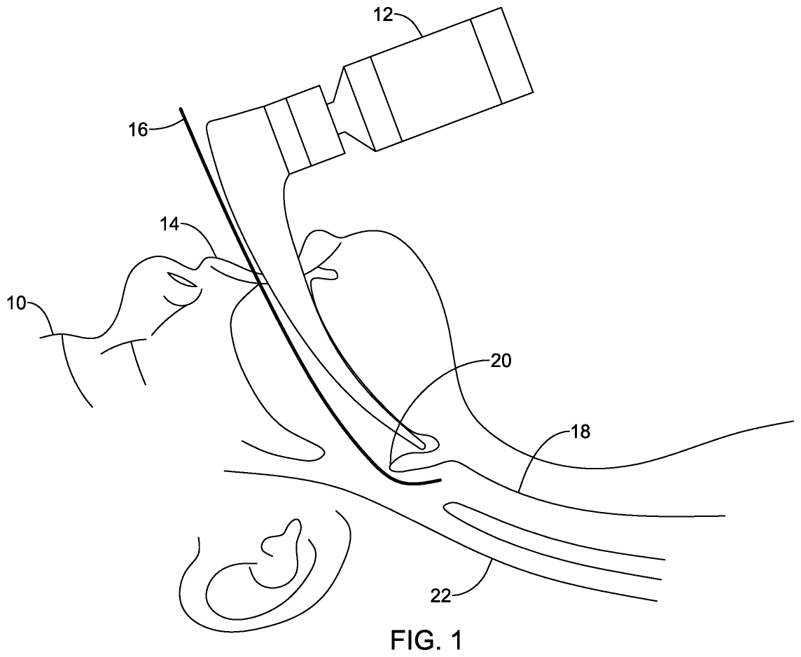

FIG. 1 depicts a schematic anatomical drawing of an illustrative subject 10 with a laryngoscope 12 inserted through a mouth 14 of the subject 10. The bolded black line 16 in FIG. 1 represents an example trajectory of an endotracheal tube into a trachea 18 of the subject 10, where an intended path of the endotracheal tube is along the laryngoscope 12, over the epiglottis 20, and into the trachea 18. Although the line 16 in FIG. 1 is depicted as passing through a mouth 14 of the subject 10, the endotracheal tube may be inserted through a nose of the subject, over the epiglottis 20, and into the trachea 18.

As can be seen from the bolded black line 16, an endotracheal tube with a curved distal tip may be useful for positioning the endotracheal tube around the epiglottis 20 and into the trachea 18 of the subject 10. Illustratively, the curvature in the bolded black line 16 demonstrates how a tip of the endotracheal tube must go over the epiglottis 20 into the trachea 18 and not take a more “natural” route that is straight into an esophagus 22 of the subject 10, which may, if taken, result in asphyxiation of the subject 10.

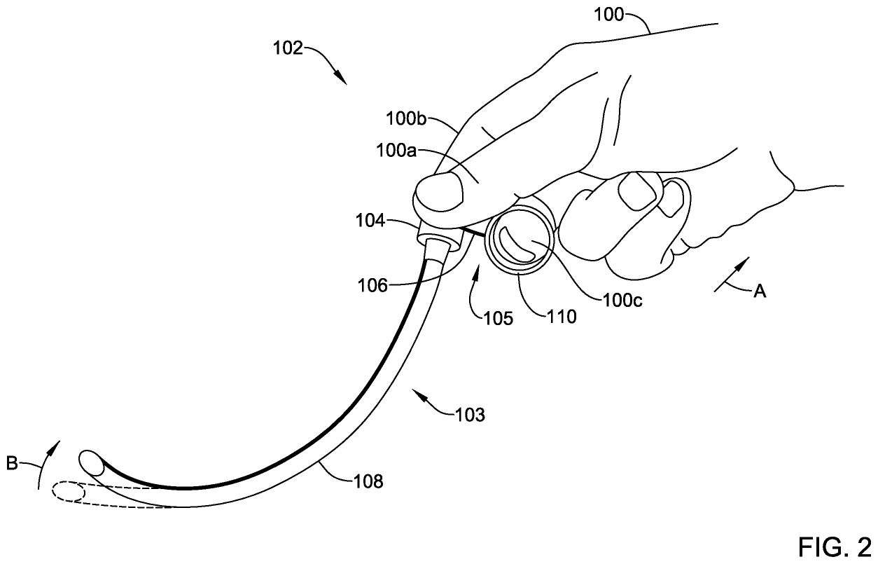

FIG. 2 depicts a user grasping an illustrative endotracheal tube assembly 102. As depicted in FIG. 2, the endotracheal tube assembly 102 may include an endotracheal tube 103 and a pull system 105.

The endotracheal tube 103 may include any suitable components. In one example, the endotracheal tube 103 may include a tube 108 and an endotracheal tube connector 104 (e.g., a standard endotracheal tube connector and/or other suitable endotracheal tube connectors used for connecting to ventilators and/or other medical systems) connected with the tube 108 (e.g., with a proximal end of the tube 108) and extending proximally from the tube 108. In some cases, the tube 108 and the endotracheal tube connector 104 may be removable from one another or adjustably connected or permanently connected. In one example configuration, the endotracheal tube connector 104 may include a stem 107 defining a lumen that is configured to be inserted into the tube 108 at a proximal end of a lumen of the tube 108 to create an adjustable or removable friction fit connection between the tube 108 and the endotracheal tube connector 104, but this is not required.

The tube 108 may be any suitable type of tube. In some cases, the tube 108 may have a single lumen and in other cases, the tube 108 may have a plurality of lumens. In one example, the tube 108 may have a first lumen configured for passing air and/or CO₂ (e.g., an airway lumen) and a second lumen (e.g., a pull line lumen) through which a pull member or line 106 of the pull system 105 may extend.

The tube 108 may formed from any suitable material. Example suitable materials for the tube 108 include, but are not limited to, resilient materials, radio-opaque material, material mixed with a radio-opaque material, a non-ferromagnetic material that does not interfere with imaging by magnetic resonance, flexible materials, plastics, pliable plastics, polyvinyl chloride (PVC), silicone, metal wrapped rubber, metal wrapped silicone, and/or other suitable materials. When the tube 108 includes an airway lumen and a pull line lumen, the pull line lumen may be formed or defined by a same or different material than is used to form or define the airway lumen. In one example, the pull line lumen may be encased by or ringed with a harder form of PVC or silicone than the material used to define the airway lumen to facilitate preventing the pull line from cutting through the material defining the pull line lumen when tension is applied to the pull line.

The endotracheal tube connector 104 may be any suitable connector having a distal end configured for engaging the tube 10 and a proximal end configured for connecting to a breathing circuit. In some cases, the endotracheal tube connector 104 may include or be coupled to a guide structure (not shown in FIG. 2, but discussed in greater detail below) to route the pull line 106 extending through and proximal of the tube 108 to a pull component 110 (e.g., a pull ring) of the pull system 105, where the guide structure may facilitate applying a curve to the tube 108 without causing rotation of the endotracheal tube 103.

The pull system 105 may include any suitable components. In one example, the pull system 105 may include the pull line 106 and the pull component 110 coupled to a proximal end of the pull line 106. In some cases, a distal end of the pull line 106 may extend to a distal end of a lumen of the tube 108 and/or couple to or with respect to a wall at a distal end of the tube 108. As discussed in greater detail below, a portion of the pull line 106 between the distal end of the tube 108 and the pull component 110 (e.g., a portion of the pull line 106 between a location the pull line 106 exits the tube 108 and the pull component 110 and/or other suitable portion of the pull line 106) may extend to and/or through (e.g., may be threaded through, etc.) a guide structure at the proximal end of the endotracheal tube assembly 102 (e.g., a guiding structure on the endotracheal tube connector 104, a guiding structure on coupled to the endotracheal tube connector 104, and/or other suitably positioned guiding structure). The guide structure may be part of the pull system 105 and/or the endotracheal tube 103.

The pull line 106 (e.g., a string or other suitable member) may be formed from any suitable type of material configured to have a tension applied thereto sufficient to bend a distal end of the tube 108. Example suitable materials include, but are not limited to, metal, polymer, plastic, nylon, polyethylene terephthalate (PET) (e.g., DACRON™, a fishing line, etc.), a wound or unwound wire (e.g., similar to strings on a string instrument and/or other suitable wound or unwound wire), and/or any other suitable materials having a desired tensile strength and that does not cut into a surface of the tube 108 (e.g., a plastic surface of a pull line conduit or lumen) at a desired curvature of the tube 108

A distal end of the pull line 106 may be positioned at and/or affixed (e.g., via adhesives, over-molding, a knot, etc.) at the tip or distal end of the tube 108. In some cases, the distal end of the pull line 106 may be affixed to an inner side of a conduit or lumen that runs within a wall of the tube 108 that has a concave outer surface, but this is not required and the pull line 106 may engage and/or be secured to the tube 108 at one or more other suitable locations. Example techniques for coupling the pull member or line 106 to the tube 108 include, but are not limited, to coupling techniques described in US Patent Application Publication No. 2020/0155807 A1, incorporated herein by reference, as discussed above.

The pull component 110 may take on any suitable form. For example, the pull component 110 may be circular, torus shaped, an elongated bar, square shaped, a rectangular shape, and/or have one or more other suitable forms. In one example, the pull component 110 may be circular or torus shaped, but this is not required.

In operation, the endotracheal tube connector 104 of the endotracheal tube assembly 102 may be held by either the user's right or left hand and with two digits of the hand of the user. A third digit may be used to engage the pull component 110, where the third digit may be flexed in the direction of arrow A to bend or otherwise apply a curvature to a distal end of a tube 108 of the endotracheal tube assembly 102 in the direction of arrow B, as depicted, for example, in FIG. 2. In one example, a user 100 may grasp the connector 104 with a thumb 100a and an index finger 100b of a hand and engage the pull component 110 with a middle finger 100c of the same hand grasping the connector 104, but this is not required and a user 100 may engage the endotracheal tube assembly 102 in one or more other suitable manners. In some cases, a hand and/or finger sizes may determine if a user engages the pull component 110 with his/her middle, ring, or small finger (e.g., pinky finger). The user 100 may flex or extend the digit engaging the pull component 110 to apply a tension to the pull line 106 and bend and/or curve the tube 108 (e.g., bend the tube to fit around an epiglottis of the subject) in response to the tension applied to the tube 108 without rotating the tube 108.

A pull or otherwise placing tension on the pull line 106 by the user 100 (e.g., a by a digit of the user) that is engaged with the pull component 110 may stiffen the tube 108 to ease rotational positioning and bend and/or lift a tip of the tube 108 over the epiglottis 20 as the tube 108 is advanced into the trachea 18 of the subject. When the tension is applied to the pull line 106, the tube 108 may curve in a direction of least resistances (e.g., in a direction of a thinnest wall of the tube 108), a direction of a pre-set curve in the tube 108, etc. As the pull member or line 106 is integrated into the pull line conduit or lumen 112 in the concave wall of the tube 108, the tube 108 remains open, or patent, throughout the intubation, and this allows for confirmation of correct tracheal intubation by auscultation of breath sounds and/or the detection of exhaled CO₂. The pull component 3 may be cut off after intubation and discarded.

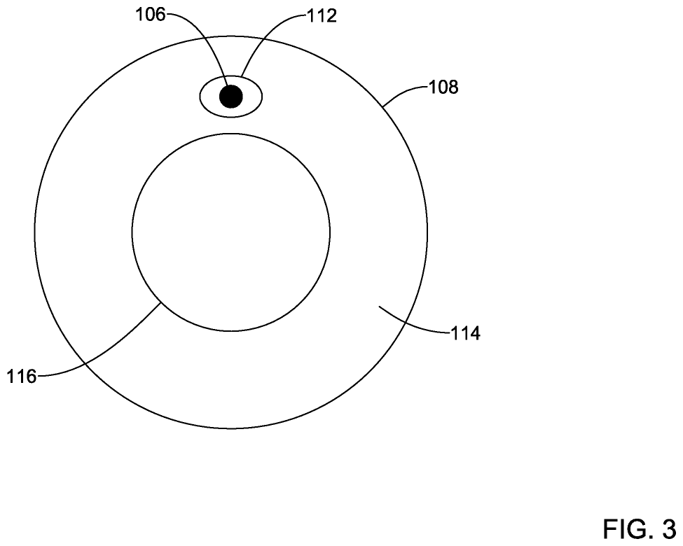

The pull line 106 may run from the pull component 110, through the guiding structure and into a lumen, which may extend along a portion of or an entirety of the tube 108. In some cases, the pull line 106 may extending within and along a pull line lumen 112 within a wall 114 of the tube 108, as depicted in the cross-sectional view of FIG. 3. In some cases, the pull line lumen 112 may extend along a portion of or an entirety of an airway lumen 116. Where the tube 108 includes a curvature (e.g., a Magill Curve and/or other suitable curvature) and/or is intended to create a curvature, the pull line lumen 112 may be located in a wall of the tube 108 at a concave side of the tube 108. The pull line lumen 112 may have an opening at a proximal terminal end and at a distal terminal end, may be closed at the distal terminal end and have an opening at the proximal terminal end, may be closed at the distal terminal end and the proximal terminal end with a side opening at a location between the terminal ends, and/or take on one or more other suitable configurations.

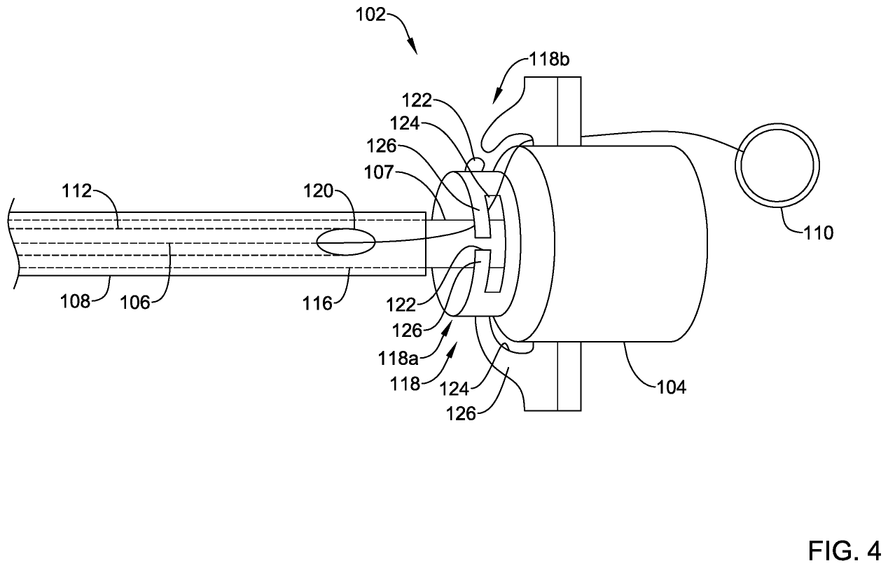

FIG. 4 depicts a partial side view of a portion of an illustrative endotracheal tube assembly 102 including a guide structure 118. The endotracheal tube assembly 102, as depicted in FIG. 4, includes the endotracheal tube 103 with the stem 107 of the endotracheal tube connector 104 coupled to a proximal end of the tube 108. The pull line 106 may extend through the pull line lumen 112 and out of a proximal side orifice or opening 120 in a wall of the tube 108, through the guide structure 118, and to the pull component 110. A proximal end of the pull line lumen 112 and the proximal opening 120 may be spaced distally a distance from a proximal end of the tube 108 that is sufficient to not compress the pull line lumen 112 when the stem 107 of the endotracheal tube connector 104 is introduced into the airway lumen 116 of the tube 108 frictionally or otherwise secured to the tube 108. Other suitable configurations of the endotracheal tube assembly 102 are contemplated.

The guide structure 118 may be any suitable guide configured to guide the pull line 106 (e.g., a proximal portion of the pull line 106 and/or other suitable portion of the pull line 106) between the tube 108 and the pull component 110 such that the pull line 106 may be tensioned without rotating the tube 108 and/or cutting through the tube 108. For example, the guide may include one or more of a hole, a slot, a groove, a channel, an opening, and/or other suitable feature configured to guide the pull line 106 received therein. In one example, the guide structure 118 may include a slot 122 that leads to a groove or channel 124 for receiving and guiding the pull line 106, as depicted in FIG. 4, but other suitable configurations are contemplated. When included, the slot 122 may be substantially straight, slanted axially and/or circumferentially, and/or take one or more other suitable shapes.

The guide structure 118 may have one or more guides configured to receive the pull line 106. For example, the guide structure 118 may include a first guide 118a and a second guide 118b, however, the first guide 118a, the second guide 118b, the first guides 118a and the second guide 118b, and/or other suitable guides or combinations thereof may be considered a guide structure 118. The illustrative endotracheal tube assembly 102 depicted in FIG. 4 includes the first guide 118a located distal of the second guide 118b, where the first guide 118a is located closer to a central axis of the endotracheal tube connector 104 than the second guide 118b, but this is not required, which may facilitate preventing or mitigating rotation of the tube 108 when a tension is applied to the pull line 106 passing through the guide structure 118.

The one or more guides of the guide structure 118 may be located along or proximate to the endotracheal tube 103 at any suitable location. For example, the guide structure 118 may be located on the endotracheal tube connector 104, distal of the endotracheal tube connector 104, in the endotracheal tube connector 104, proximal of the endotracheal tube connector 104, and/or at one or more other suitable location. As depicted in FIG. 4, the guide structure 118 may be located at the endotracheal tube connector 104 in one example.

When positioned at or proximate a component of the endotracheal tube assembly 102, the guide structure 118 may be configured to extend partially or entirely around the component of the endotracheal tube assembly 102. In the example depicted in FIG. 4, the guide structure 118 may include the slot 122 leading to the channel 124 that extends partially around a circumference of the endotracheal tube connector 104. When two or more guides are utilized, the guides may be axially and/or circumferentially offset from one another. As depicted in FIG. 4, the first guide 118a may extend along a first circumferential portion of the endotracheal tube connector 104 and the second guide 118b may extend along a second circumferential portion of the endotracheal tube 108 that is partially or entirely opposing the first guide 118a, but this is not required. Although not required, the first guide 118a may be aligned with the proximal opening 120 on the tube 108 to facilitate the pull line 106 extending out of the proximal opening to the first guide 118a in a straight or substantially straight manner and the second guide 118b may be located at an opposite side of the endotracheal tube assembly 102 from a side at which the proximal opening 120 is located.

In some cases, the first guide 118a and/or the second guide 118b may include a semi-ring along the endotracheal tube connector 104. The channels 124 of the first and/or second guides 118a, 118b may extend around the endotracheal tube connector 104 in circumferentially opposite directions from the slit or slot 122. As depicted in FIG. 4, the channels 124 (e.g., bilateral channels) of the first guide 118a may be located at the base of the endotracheal tube connector 104 and extend an equal circumferential distance from the slot 122, but this is not required. The channels 124 may be at least partially defined by arms of the first and/or second guides 118a, 118b 126 (e.g., an outer periphery of the guides 118a, 118b) and the endotracheal tube connector 104. In some cases, the channels 124 and the arms 126 may be or may be part of a channel structure configured to receive the pull line 106, where the channel structure is attached to and/or integrated into the endotracheal tube connector 104.

In operation of the endotracheal tube assembly 102 depicted in FIG. 4, the pull line 106 extending out of the proximal opening 120 may be threaded into the first guide 118a on the endotracheal tube connector 104 and then through the second guide 118b. The slot 122 between the arms 126 may facilitate threading the pull line 106 into the channels 124 to allow for the pull line 106 to be threaded through the guide structure 118. The channels 124 of the first guide 118a may facilitate guiding the pull line 106 extending out of the pull line lumen 112 at the proximal opening 120 to the second guide 118b located on or proximate to a side (e.g., a second side) of the endotracheal tube connector 104 opposite of or circumferentially spaced from a side (e.g., a first side) of the endotracheal tube connector 104 at which the channels 124 of the first guide 118a are located. Further, the first guide 118a may maintain the pull line 106 in a relatively straight line between an end of the pull line lumen 112 and/or the proximal opening 120 and the channels 124 of the first guide 118a due, at least in part, to a proximity of the first guide 118a to the central axis of the endotracheal tube connector 104 such that the pull line 106 does not twist the tube 108, or twisting of the tube is mitigated, when a tension is applied to the pull line 106.

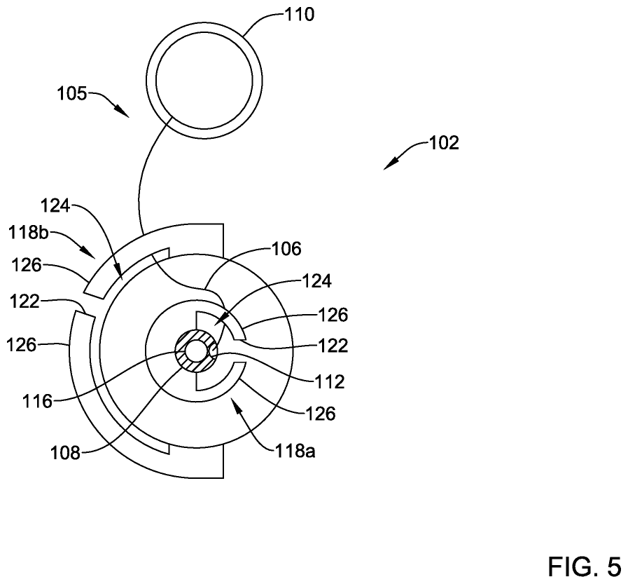

FIG. 5 depicts a schematic distal end view of the illustrative endotracheal tube assembly 102 depicted in FIG. 4, with a section of the tube 108 distal of the proximal opening 120 removed to depict a cross-section of the tube 108 with the pull line 106 extending through the pull line lumen 112. A portion of the pull line 106 extending proximally from the proximal opening 120 may be threaded through the first guide 118a to the second guide 118b, with a proximal end of the pull line 106 coupled to the pull component 110. As discussed above, threading the pull line 106 through the first guide 118a and the second guide 118b may facilitate pulling the pull line 106 extending out of the proximal opening 120 toward the endotracheal tube connector 104 in a straight or substantially straight line from the tube 108 to the first guide 118a, so as to not twist the tube 108 while the pull line 106 and/or the tube 108 are under tension or traction in response to a user pulling on the pull component 110 or otherwise applying tension to the pull member or line 106.

As depicted in FIG. 5, the arms 126 of the first guide 118a and the second guide 118b may define a semiring that extends or protrudes from the endotracheal tube connector 104 and/or may take on one or more other suitable configurations. In one example, the arm 126 of the first guide 118a and the second guide 118b may protrude laterally from opposing sides of the endotracheal tube connector 104, with the second guide 118b protruding from a side of the endotracheal tube connector 104 opposite or substantially opposite a side of the endotracheal tube connector 104 at which the concave bend of the tube 108 is directed when tension or traction is applied to the pull member or line 106, but this is not required.

The slots 122 of the guide structure 118 may be centered along a guide or offset from a center of a guide. In the example depicted in FIG. 5, the slot 122 of the first guide 118a is centered such that the arms 126 of the first guide 118a have equal or substantially equal lengths and the slot 122 of the second guide 118b is offset from a center of the guide such that one arm 126 of the second guide 118b is shorter than the other arm 126. However, the configuration depicted in FIG. 5 is not required and other suitable positions of the slot 122 are contemplated.

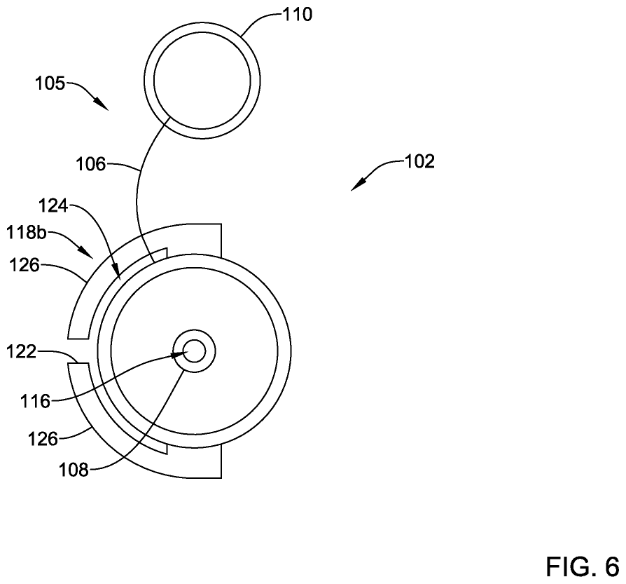

FIG. 6 depicts a top view of an illustrative endotracheal tube assembly 102. The endotracheal tube assembly 102 depicted in FIG. 6 is similar to the endotracheal tube assembly 102 depicted in FIGS. 4 and 5, but with the slot of the second guide 118b centered such that the arms 126 of the second guide 118b have equal or substantially equal lengths.

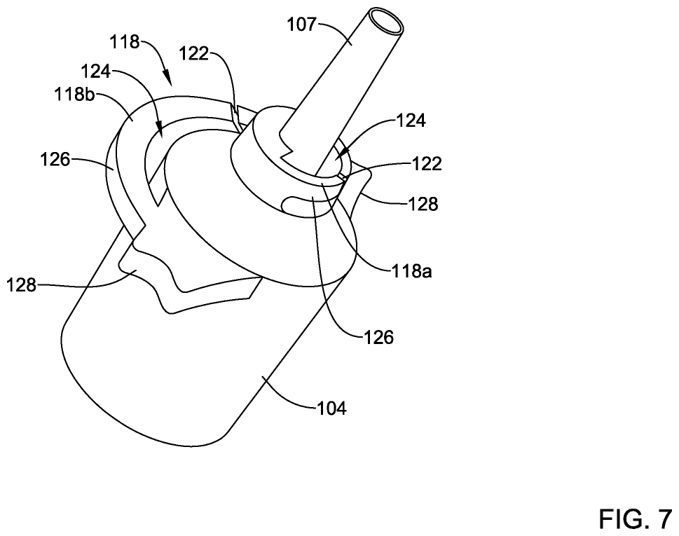

FIG. 7 depicts a schematic bottom perspective view of an illustrative configuration of the endotracheal tube connector 104. As depicted in FIG. 7, the slot 122 may be offset from a center of the second guide 118b. In some case, the endotracheal tube connector 104 may include bilateral attachment knobs or rings 128. As depicted in FIG. 7, the endotracheal tube connector 104 may include two bilateral attachment knobs or rings extending radially outward relative to a central axis of the endotracheal tube connector 104 in opposite directions. However, the bilateral attachment knob or ring 128 may take on one or more other suitable configurations or may be omitted, as desired. When included and although not required, the bilateral attachment knob or ring 128 may be axially aligned with (e.g., at a same or similar axial location as) the second guide 118b.

In operation of the endotracheal tube assemblies depicted in FIGS. 4-7B, when the user engages the pull component 110 with her/his middle finger (or other finger) and flexes that middle figure, the pull component 110 may exert tension and/or traction on the pull line 106 that is externally threaded in the first guide 118a, around the endotracheal tube connector 104, and externally threaded through the second guide 118b such that the tube 108 may be tensioned to a concave curvature. The distal tip of the pull line 106 may be affixed to the tube 108 within the pull line lumen 112 at the distal end (e.g., at the distal tip or other distal end location) of the tube 108, as discussed above. The tension and/or traction on the pull line 106 may result in stiffening of the tube 108 and the curving of a portion of the tube 108 that may be configured to be introduced over the epiglottis 20 and into the trachea 18, in order to avoid intubation of a subject's esophagus 22, which could have the serious and dangerous effect of asphyxiating the subject.

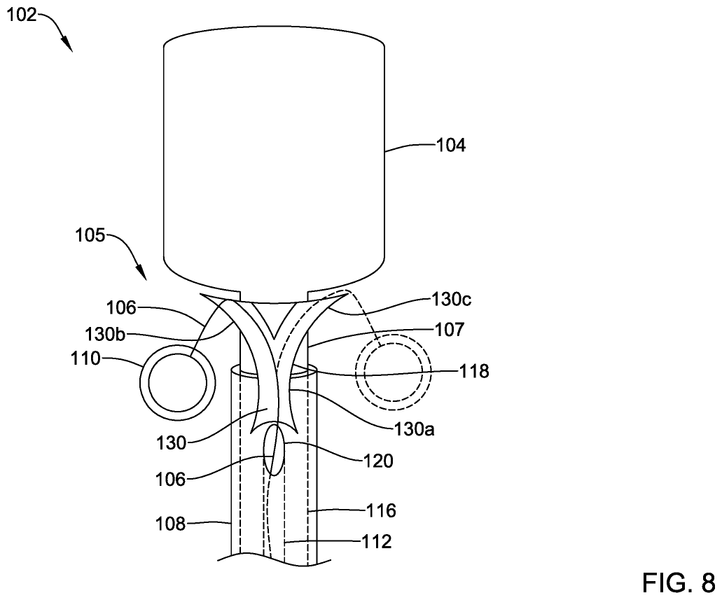

FIG. 8 depicts an illustrative the endotracheal tube assembly 102 with the guide structure 118 having a ramped configuration. As depicted in FIG. 8, the pull line 106 may extend through a ramp or groove 130 of the guide structure 118.

The ramp or groove 130 may have any suitable size and/or shape. In one example, as depicted in FIG. 8, the guide structure 118 may extend to the proximal opening 120 and may include a Y-shaped ramp or groove that includes a central portion 130a extending proximally from proximate the proximal opening 120, a first portion 130b branching toward a first direction from the central portion 130a and a second portion 130c branching toward a second direction.

The pull line 106, as depicted in FIG. 8, may extend out of the proximal opening 120, through the central portion 130a of the groove 130 in the guide structure 118 and along one of the first portion 130b or the second portion 130c of the groove 130 such that a tension may be applied to the pull line 106 without rotating the tube 108. In some cases, the pull line 106 may be threaded through the first portion 130b of the groove 130 for a right-handed user to easily engage the pull component 110. An imaginary pull line 106 threaded in the second portion 130c of the groove 130 for easy engagement of the pull component 110 by a left-handed user is depicted in broken lines.

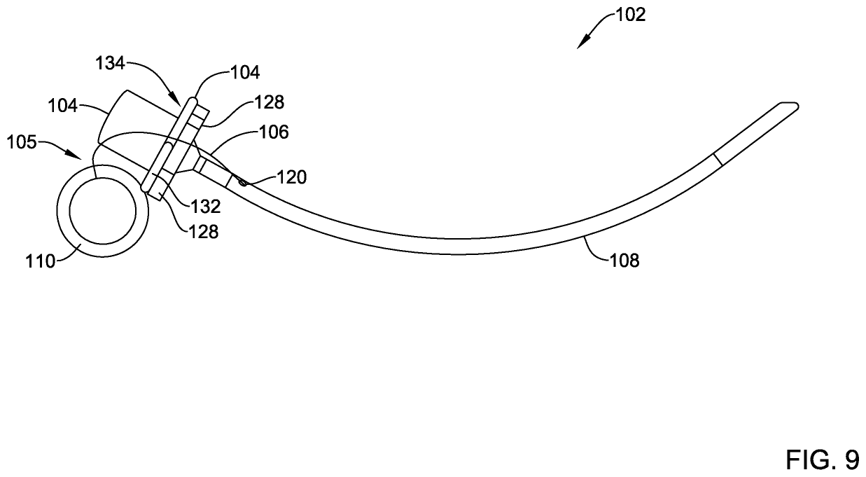

FIG. 9 depicts a schematic view of an illustrative endotracheal tube assembly 102 having the guide structure 118, where the guide structure 118 is defined by a guide component 132. The guide component 132 depicted in FIGS. 9-11 may be configured to be retrofitted onto an endotracheal tube connector 104, but this is not required.

The guide component 132 may include a pull line receiving portion 134 configured to receive and guide a portion of the pull line 106 extending proximally from the tube 108. In some cases, the pull line receiving portion 134 may be aligned with the proximal opening 120 of the tube 108 such that the pull line 106 extends in a straight or substantially straight line between the proximal opening 120 and the guide structure 118 and/or the endotracheal tube connector 104 to facilitate applying a straight concave bend or curvature to the tube 108 when a tension is applied to the pull line 106.

The guide component 132 may have any suitable shape and size. In some cases, the guide component 132 may have a rounded or circular portion and may be disc-shaped. The guide component 132, however, may have other suitable configurations configured to couple to the endotracheal tube connector 104 and receive the pull line 106.

In some cases, the guide component 132 may be configured to be applied over a proximal end of the endotracheal tube connector 104 and rest against a proximal side of the bilateral attachment knobs 128. Alternatively or additionally, the guide component 132 may be configured to be applied over a distal end of the endotracheal tube 103 and rest against a distal side of the bilateral attachment knobs 128.

The guide component 132 may be secured to the endotracheal tube connector 104 in any suitable manner. For example, the guide component 132 may be secured to the endotracheal tube connector 104 via a luer lock, a friction fit, an adhesive, a detent coupling, and/or one or more other suitable types of connection. In one example, the guide component 132 may be secured to the endotracheal tube connector 104 via an adhesive. In another example, the guide component 132 may be secured to the endotracheal tube connector 104 via a friction fit.

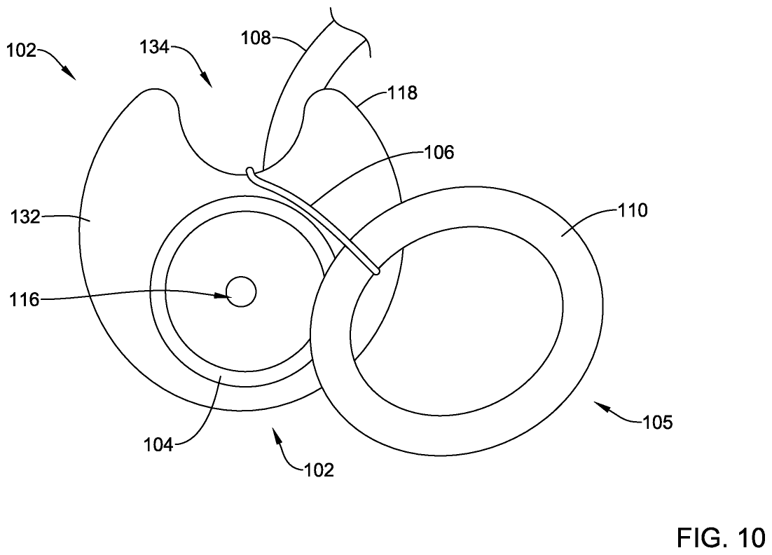

FIG. 10 depicts a schematic top view of the illustrative endotracheal tube assembly 102 depicted in FIG. 9. As depicted in FIG. 10 the pull line receiving portion 134 of the guide component 132 may be formed from a recess or opening defined by an outer circumference or extent of the guide component 132. The recess or opening may have any suitable shape and/or size for receiving the pull line 106 and facilitating use of the endotracheal tube assembly 102 with a left hand of a user and a right hand of the user. In one example, the receiving portion 134 may be trough shaped with a circumferentially wider radially outer extent than a width of a radially inner extent, as depicted in FIG. 10. Further, although the receiving portion 134 is shown with a rounded inner extent, this is not required and other suitable configurations are contemplated.

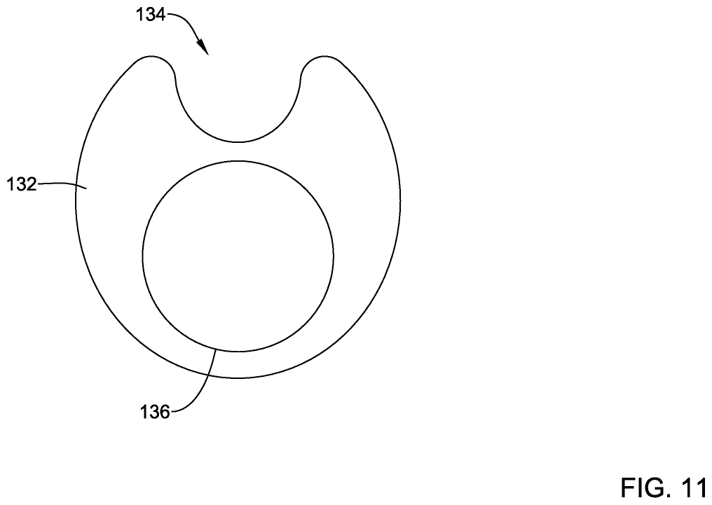

FIG. 11 depicts a top view of the illustrative guide component 132 of or attached to the endotracheal tube assembly 102 depicted in FIGS. 9 and 10. The guide component 132 includes the pull line receiving portion 134, as discussed, and a connector receiving portion 136 configured to receive the endotracheal tube connector 104 and/or the tube 108 of the endotracheal tube assembly 102. To facilitate receiving the endotracheal tube connector 104, the connector receiving portion 136 may have a similar shape to a portion of the endotracheal tube connector 104 to be received within the connector receiving portion 136 and may be sized slightly smaller than, the same size as, or slightly larger than the portion of the endotracheal tube connector 104 the connector receiving portion 136 is configured to receive. In some cases, the connector receiving portion 136 may be circular or substantially circular. In some cases, the connector receiving portion 136 and the endotracheal tube connector 104 may be keyed relative to each other to ensure a desired orientation of the guide component 132 relative to the endotracheal tube connector 104 and/or the tube 108.

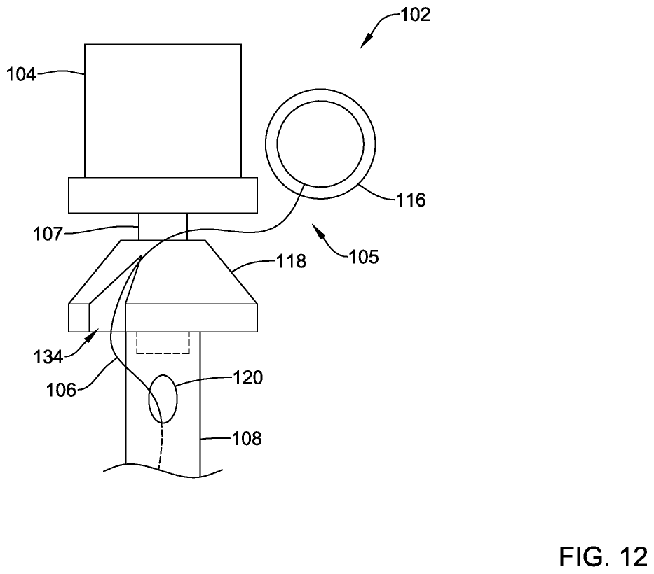

FIG. 12 depicts a schematic view of an illustrative endotracheal tube assembly 102 configuration having the guide structure 118. In some cases, the guide structure 118 may be located at or may extend distally from a distal end of the endotracheal tube connector 104. Alternatively or additionally, the guide structure 118 may be part of the endotracheal tube connector 104 and form the distal end or other suitable portion of the endotracheal tube connector 104. In one example, the guide structure 118 may be secured to or at the stem 107 of the endotracheal tube connector 104. Although not required, in some cases, the guide structure 118 may be fully or at least partially secured in a desire position by a proximal end of the tube 108 engaging a distal side of the guide structure 118 and a distal end of a base of the endotracheal tube connector 104 engaging a proximal side of the guide structure 118.

The guide structure 118 of the illustrative endotracheal tube assembly 102 configuration depicted in FIG. 12 may have any suitable size and/or shape. For example, the guide structure 118 may have a conical shape, a frustoconical shape, a taper toward a proximal end of the endotracheal tube assembly, and/or one or more other suitable shapes or configurations. In one example configuration, the guide structure 118 may have a conical ring configuration that tapers toward the proximal end of the endotracheal tube assembly 102, but this is not required.

In some cases, the guide structure 118 may include the pull line receiving portion 134 (e.g., an indent or other suitable configuration) configured to receive the pull line 106 extending proximally from proximal opening 120 of the tube 108. In one example, the pull line receiving portion 134 of the guide structure 118 in the endotracheal tube assembly 102 depicted in FIG. 12 may be a V-shaped slit, but this is not required and the pull line receiving portion 134 may have one or more other suitable configurations.

In operation of the endotracheal tube assembly 102 configuration depicted in FIG. 12, the pull line 106 having a proximal end coupled to the pull component 110 may be guided through the pull line receiving portion 134, under the base of the endotracheal tube connector 104, and to an opposite side (e.g., a second side) of the endotracheal tube assembly 102 (e.g., an opposite side of the endotracheal tube connector 104 and/or the tube 108) from a side (e.g., a first side) at which the pull line 106 exits the tube 108, which may facilitate positioning the pull component 110 proximate a user's digit to be used to apply tension to the pull wire 106. A pulling force by a user on the pull component 110 coupled to pull line 106 extending through the pull line receiving portion 134 of the guide structure 118 may facilitate stiffening and/or bending of the tube 108 into a concave form for insertion around an epiglottis of a subject, as discussed herein or otherwise.

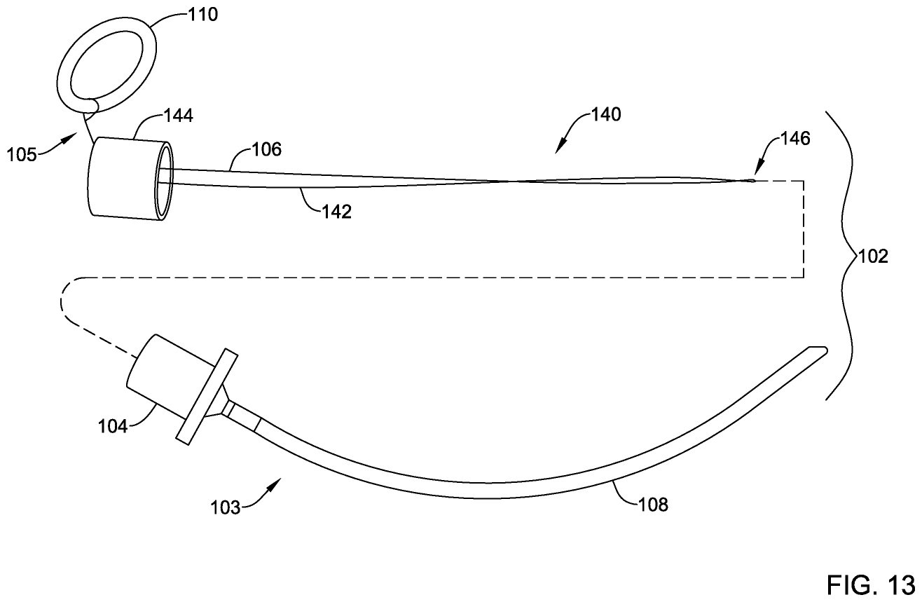

FIG. 13 depicts a schematic view of an illustrative endotracheal tube assembly 102 including an endotracheal tube 103 and an endotracheal tube adaptor 140. The endotracheal tube 103 may have an endotracheal tube connector 104 and a tube 108, similar to or different from as discussed above, where the tube 108 may have a pre-set bend. The endotracheal tube adaptor 140 may be configured to be applied to the endotracheal tube connector 104 and extend into or otherwise along the tube 108. The endotracheal tube adaptor 140 may be configured to manipulate a positioning of the tube 108 when the endotracheal tube adaptor 140 is coupled to the endotracheal tube 103. In some cases, the endotracheal tube assembly 102 may be configured for use with a single hand, such that a second hand of the user may be utilized for other purposes facilitating an intubation (e.g., to hold laryngoscope and/or for one or more other suitable purposes).

The endotracheal tube adaptor 140 may have any suitable configuration. In some cases, the endotracheal tube adaptor 140 may have the pull system 105 that includes the pull line 106, a compression component 142, and the pull component 110, along with a cap 144. The pull component 110 may be coupled to a proximal end of the pull line 106 at a distance spaced from the cap 144 sufficient to allow a user to grasp the cap 144 and the pull component 110 with digits of a single hand. When forming the endotracheal tube assembly 102 with the endotracheal tube 103, the pull line 106 and the compression component 142 may be inserted into a lumen of the tube 108 (e.g., an airway lumen and/or other suitable lumen of the tube 108 as discussed herein or otherwise) and the cap 144 may be engaged with the endotracheal tube connector 104.

In some cases, the distal ends of the pull line 106 and the compression component 142 may be connected and form a combined distal end 146. The distal end 146 of the pull line 106 and the compression component 142 may be a blunt distal end, but this is not required.

The pull line 106 and/or the compression component 142 may be referred to as steering elements. Additionally or alternatively, the pull line 106 and compression component 142 may be a single component and referred to collectively as an adjustment member, where the compression component 142 may be a first portion extending from a first end of the adjustment member, the pull line 106 may be a second portion extending from a second end of the adjustment member. The adjustment member may include a bend at a distal end of the first portion and a distal end of the second portion, where the bend may couple and/or extend between the first portion and the second portion. The first end of the adjustment member may be coupled to a holder of the cap 144 (e.g., as discussed below with respect to the compression component 142) and the second end of the adjustment member may be coupled to the pull component 110 (e.g., as discussed herein with respect to the pull line 106).

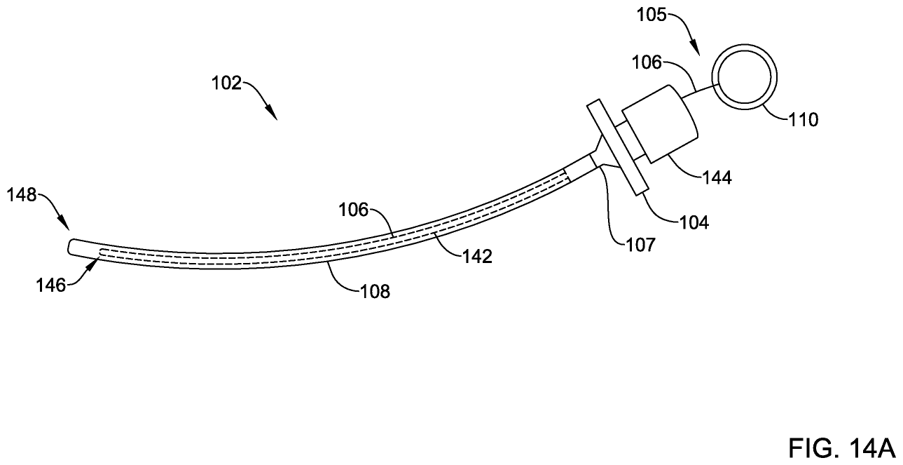

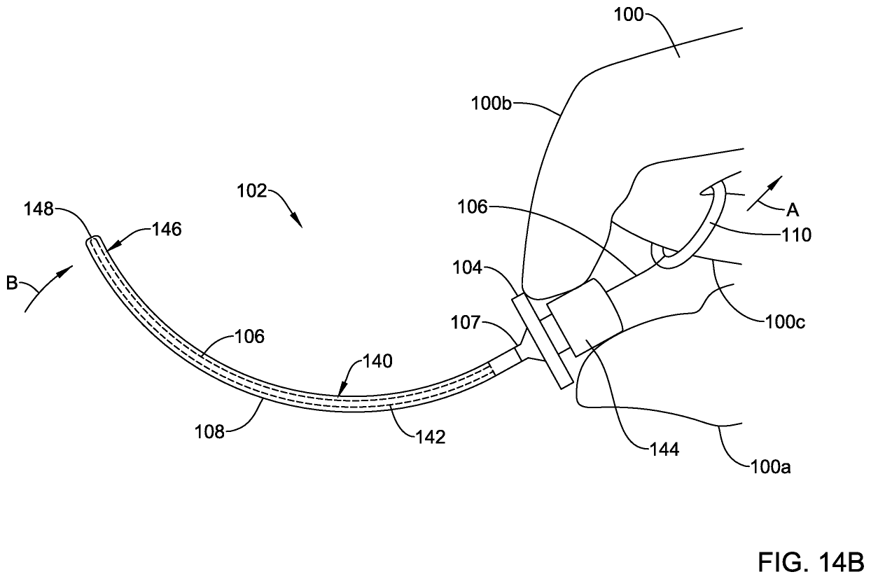

FIGS. 14A and 14B depict schematic views of the illustrative endotracheal tube assembly 102 depicted in FIG. 13 with the endotracheal tube adaptor 140 inserted into and coupled to the endotracheal tube 103. As depicted in FIG. 14A, the pull line 106 and the compression component 142 may be inserted into the tube 108 and the cap 144 may be applied to the endotracheal tube connector 104. When inserted into the tube 108, the distal end 146 formed by pull line 106 and the compression component 142 may be positioned adjacent a distal end of the tube 108, but at or proximal of a distal opening 148 in the tube 108.

The cap 144 may be coupled to the endotracheal tube connector 104 in any suitable manner. For example, the cap 144 may be coupled to the endotracheal tube connector 104 using a friction fit, a squeeze fit (e.g., via a user squeezing the cap 144 against the outer surface of the endotracheal tube connector 104, etc.), a threaded connection, a snap fit connection, a luer lock connection, and/or other suitable types of connections or couplings. In one example, the cap 144 may be coupled to the endotracheal tube connector 104 via a friction fit, as depicted in FIG. 14A. In some cases, however, the cap 144 may be spaced from or otherwise not coupled to the endotracheal tube connector 104, while the pull component 110 and pull line 106 are actuated by a user to manipulate a position of the tube 108.

FIG. 14B depicts a user 100 grasping a proximal end of the endotracheal tube assembly 102, where the thumb 100a and index finger 100b of the user 100 is grasping the endotracheal tube connector 104 and/or the cap 144. While grasping the endotracheal tube connector 104 and/or the cap 144, the middle finger 100c or other finger of the user 100 may engage the pull component 110. As the user flexes its middle finger 100c and/or otherwise pulls on the pull component 110 in the direction of arrow A, the distal end of the tube 108 moves in the direction of arrow B. In operation, the movement of the pull component 110 in the direction of arrow A, pulls the pull line 106 proximally while the compression component 142 maintains the distal end 146 of the pull line 106 and the compression component 142 within the tube 108. When the pull line 106 is pulled, the distal end 146 of the pull line 106 and the compression component 142 may apply a force on the tube 108 and cause the tube 108 to bend further in the direction of the pre-set bend (e.g., in the concave direction) to a desired position. Such ability to control a position of the distal end of the tube 108 facilitates entering a subject's trachea with the tube when maneuvering around the subject's epiglottis, rather than entering the esophagus of the subject and potentially asphyxiating the subject.

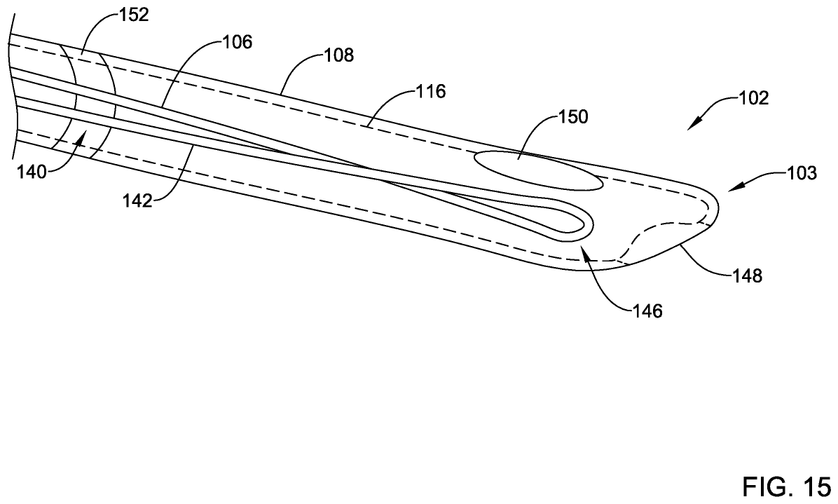

FIG. 15 depicts a schematic view of a distal end of the endotracheal tube assembly 102 depicted in FIG. 13, with the endotracheal tube adaptor 140 coupled with the endotracheal tube 103. As depicted in FIG. 15, when the endotracheal tube adaptor 140 is coupled with the endotracheal tube 103, the distal end 146 of the pull line 106 and the compression component 142 remains within the tube 108. In some cases, the pull line 106 and the compression component 142 may travel through the tube 108 in parallel, but this is not required and the pull line 106 and the compression component 142 may cross or intersect at one or more points along the tube 108.

The distal end of the tube 108 may have any suitable configuration. In one example, the tube 108 may have a beveled distal end defining the distal opening 148, with a side opening 150 (e.g., a Murphy's eye or other suitable side opening) located in a side wall and positioned proximal of distal opening 148. In some cases, the side opening 150 may function as a vent and may prevent a complete obstruction of the subject's airway should the distal opening 148 become occluded. The side opening 150, however, may be omitted in some cases. Further, although not required, the tube 108 may have one or more distance markers 152 that facilitate a user understanding a position of the tube 108 within a subject.

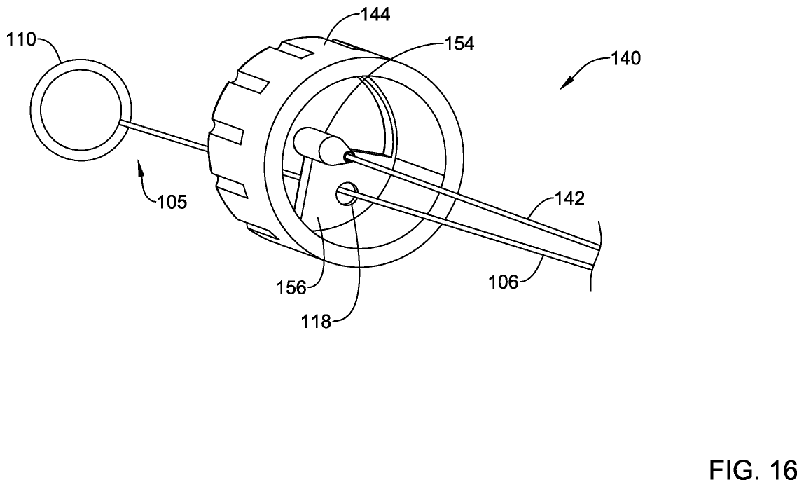

FIG. 16 depicts a schematic bottom perspective view of a portion of the endotracheal tube adaptor 140. The endotracheal tube adaptor 140 may include, among other features, the pull line 106, the compression component 142, the cap 144, a holder 154, a plate 156, and the guide component 118.

Similar to as discussed above with respect to the pull line 106, the pull line 106 and/or the compression component 142 may be formed from any suitable type of material configured to have a tension applied thereto sufficient to bend a distal end of the tube 108 of the endotracheal tube 103. Example suitable materials include, but are not limited to, metal, polymer, plastic, nylon, polyethylene terephthalate (PET) (e.g., DACRON™, a fishing line, etc.), a wound or unwound wire (e.g., similar to strings on a string instrument and/or other suitable wound or unwound wire), and/or any other suitable materials having a desired tensile and/or compressive strength and bendability and that do not cut into a surface of the tube 108 at a desired curvature of the tube 108.

The cap 144 of the endotracheal tube adaptor 140 may be formed from any suitable material. Example suitable materials include, but are not limited to, metal, polymer, plastic, nylon, polyethylene terephthalate (PET), an opaque material, a transparent material, and/or any other suitable materials. In some cases, at least a portion of the cap 144 may be formed from a transparent polymer. The material of the cap 144 may be configured to withstand forces applied thereto by pulling on the pull component 110 and engagement of the cap with the endotracheal tube connector 104, while engaging and guiding the steering elements (e.g., the pull line 106 and the compression component 142).

The cap 144 may have any suitable shape and/or configuration. In some cases, the cap 144 may have an inner diameter configured to fit over the endotracheal tube connector 104 of an endotracheal tube 103. In one example, the inner diameter of the cap 144 may taper in a proximal direction and/or otherwise be configured to create a friction fit with the endotracheal tube connector 104.

The plate 156 may extend radially inward from an inner surface of a wall of the cap 144 defining the inner diameter. The plate 156 may have any suitable configuration for supporting the compression component 142 and the holder 154 and/or for guiding the pull line 106. In some cases, a size of the plate 156 may be sized to provide adequate support and guidance to the steering elements, while allowing space for the air and CO₂ to pass through the cap 144 to and from the subject. As depicted in FIG. 16, the plate 156 may be or may have a triangular or circular sector configuration, but this is not required.

The holder 154 may have any suitable configuration for supporting the compression component 142. In one example, the holder 154 may be elongated and coupled to the compression component 142, which may prevent or mitigate buckling of the compression component 142 at or near the holder 154. The holder 154 may be coupled to the compression component in any suitable manner. In some cases, the holder 154 may be coupled to and/or secured to a proximal end of the compression component 142 by over molding the holder 154 onto the compression component 142, but this is not required and other suitable coupling techniques are contemplated.

The holder 154 may be positioned at any suitable location within the cap 144. In one example, the holder 154 may be located in a center (e.g., at a central axis of) or a middle of the cap 144 above and/or aligned with an airway opening within the endotracheal tube connector 104 and in communication with an airway lumen of the tube 108 to allow air and/or CO₂ to pass through the cap 144. In some cases, the holder 154 may be supported within the cap 144 by the plate 156 extending from a wall of the cap 144 and located radially inward from the guide 118 in the plate 156 such that the guide 118 may be radially outward from the holder 154. The holder 154 may be spaced proximally from the airway opening within the endotracheal tube connector 104 to allow space for air and/or CO₂ to pass out of the endotracheal tube 103. When the plate 156 has a triangular or circular sector configuration, the holder 154 may be formed by or secured to a central-most portion of the plate 156, but this is not required.

The endotracheal tube adaptor 140 may include a CO₂ detector. In one example, the holder 154 and/or the plate 156 (e.g., an underside of the plate 156 or other suitable portion of the plate 156) may include or may be covered with a color changing CO₂ detection material (e.g., a pH indicator or other suitable detector), but this is not required. Other suitable CO₂ detection features are contemplated. In some cases, the holder 154 and the guide 118 may be positioned within the cap 144 to allow the user to view the CO₂ detector, when included, and/or to detect breathing of the subject in one or more other suitable manners including, but not limited to, viewing bubbles coming out of or being sucked into the airway opening 158, hearing moved air or carbon dioxide, and/or other suitable indicators of successful or unsuccessful intubation.

The endotracheal tube adaptor 140 may include a guide structure 118. Similar to as discussed above, the guide structure 118 may be any suitable guide configured to guide the pull line 106 between the tube 108 and the pull component 110. For example, the guide structure 118 may be or may include one or more of a hole, a slot, a groove, a channel, an opening, and/or other suitable component configured to guide the pull line 106 received therein. In one example, the guide structure 118 may include a circular hole in or otherwise extending through the plate 156, as depicted in FIG. 16, but other suitable configurations are contemplated.

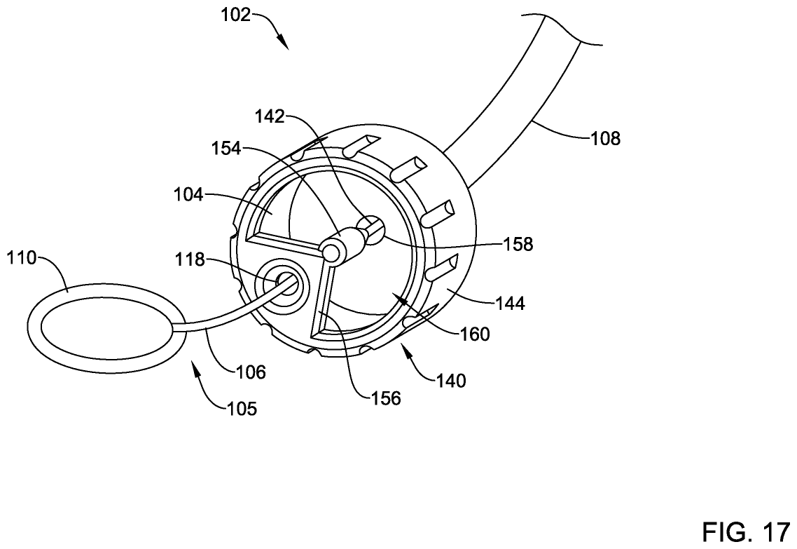

FIG. 17 depicts a schematic proximal perspective view of the endotracheal tube assembly 102. As depicted in FIG. 17, the holder 154 is aligned with an airway opening 158 in the endotracheal tube connector 104 and the compression component 142 extends from the holder 154 through the airway opening 158. The pull line 106 extends from the pull component 110, through the guide structure 118, and into the airway opening 158 proximate to the compression component 142. Air and/or CO₂ may pass to and/or from the airway opening 158 and through an opening 160 at least partially defined by the plate 156 and/or the cap 144.

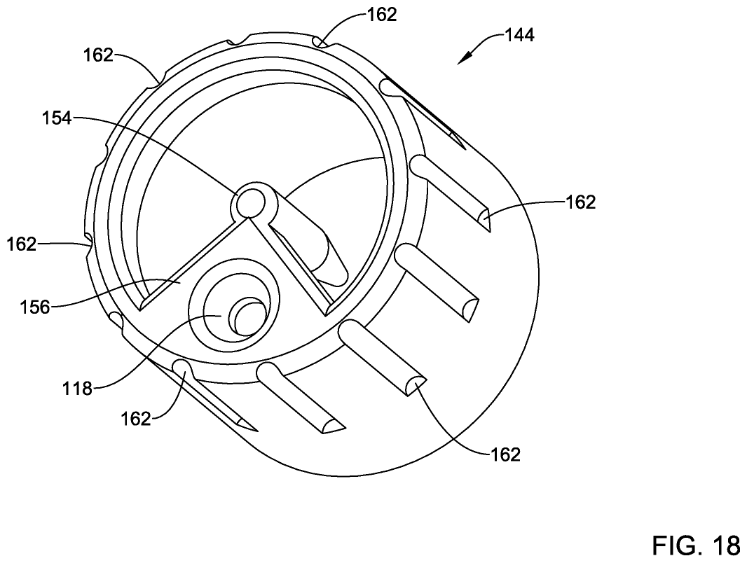

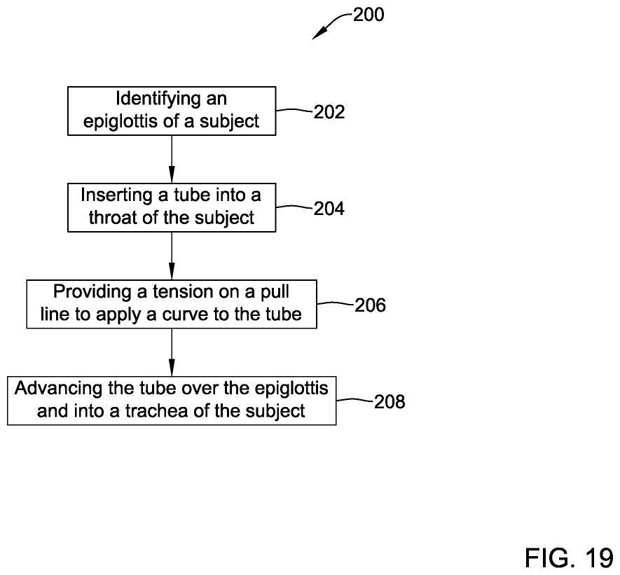

FIG. 18 depicts a schematic proximal perspective view of the cap 144 including the holder 154, the plate 156, and the guide structure 118 as a monolithic structure. In some cases, the guide structure 118 may include a proximal end that facilitates movement of the pull line 106 within the guide structure 118 while mitigating stress on the pull line 106 from a proximal edge of the guide structure 118.

Further, an exterior surface of the cap 144 may include one or more grip features 162. As depicted in FIG. 18, the grip features 162 may include elongated indents in an outer surface of the cap 144, but other suitable grip features 162 are contemplated and include, but are not limited to, protrusions, dimples, and/or other suitable features configured to facilitate gripping the cap 144 with digits of a user's hand.

The endotracheal tube assembly 102 discussed herein and/or other suitable endotracheal tube assemblies 102 may be used in an intubation method 200. The method 200 may include identifying 202 an epiglottis of a subject. In some cases, the epiglottis of a subject may be identified by holding a laryngoscope in a first hand and shining a light into a subject's throat.

Once the subject's epiglottis is identified, the user may insert 204 a tube of an endotracheal tube assembly into the subject's throat, via a mouth or nose of the subject, to the identified epiglottis. During insertion of the tube into the throat of the subject, the tube may have a pre-set or resting bend condition. In some cases, the user may grasp the endotracheal tube assembly with a second hand and insert the endotracheal tube assembly with the second hand while holding the laryngoscope with the first hand. The user may grasp the proximal end of the endotracheal tube assembly with two digits (e.g., a thumb and index figure or other suitable digits) of the second hand.

Upon reaching the epiglottis of the subject, the user may provide 206 a tension to a pull line extending through a guide of the endotracheal tube assembly to apply a curve to the tube in the direction of a trachea of the subject and advance 208 the tube distally over the epiglottis and into the subject's trachea. To bend the tube, the user may apply a pulling force on a pull component connected to a pull line extending through a guide structure and into the tube, such that different amounts of pulling force on the pull line determines an amount of bend in the tube. Once the tube is in the trachea of the subject, the user may release the pulling force on the pull component and confirm an airway is formed between the trachea of the subject and an exterior of the subject.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A tube assembly, comprising:
a tube having a distal end and a proximal end;
a pull line extending through the tube; and
a guide configured to guide a proximal portion of the pull line at a location proximal of the tube, and
wherein the pull line is configured relative to the tube and the guide such that when tension is applied to the pull line, the tube bends in response to the tension applied to the pull line to form a curvature in the tube.

2. The tube assembly of claim 1, further comprising:
a connector coupled to the proximal end of the tube.

3. The tube assembly of claim 2, wherein the guide comprises a first guide located at a first circumferential location along the connector and a second guide located at a second circumferential location along the connector, the second circumferential location is at a side of the connector opposite a side at which the first circumferential location is positioned.

4. The tube assembly of claim 2, further comprising:
a cap configured to engage the connector, and
wherein the cap defines the guide.

5. The tube assembly of claim 4, further comprising:
a compression component, and
wherein a first end of the compression component is secured to the cap, a second end of the compression component is coupled to the pull line, and the pull line is configured to extend through the guide defined by the cap.

6. The tube assembly of claim 1, wherein the guide comprises a first guide and a second guide, the first guide is located distal of the second guide.

7. The tube assembly of claim 1, wherein the guide comprises a first guide and a second guide, the first guide is circumferentially offset from the second guide.

8. The tube assembly of claim 1, further comprising:
a pull component coupled to a proximal end of the pull line; and
wherein the pull component and the pull line are configured such that the tension is applied to the pull line when a user pulls on the pull component.

9. The tube assembly of claim 1, wherein:
the tube comprises an airway lumen and a pull line lumen,
a distal end of the pull line is coupled to the tube at a location within the pull line lumen, and
the pull line exits the tube at a side orifice in communication with a proximal end of the pull line lumen.

10. The tube assembly of claim 1, wherein the guide is configured to guide the pull line in a direction proximal from an orifice in a first side of the tube to a location on a second side of the tube.

11. The tube assembly of claim 1, further comprising:
a connector coupled to the proximal end of the tube; and
wherein the guide comprises a frustoconical ring extending around the connector and the frustoconical ring includes a slit opening toward the first side of the tube, the slit is configured to receive the pull line and when tension is applied to the pull line.

12. The tube assembly of claim 1, further comprising:
a cap defining the guide; and
a compression component, and
wherein:
the compression component is secured to the cap and the pull line extends through the guide defined by the cap; and
a bend is located between the compression component and the pull line such that the compression component extends between a location at which the compression component is secured to the cap and the bend and the pull line extends from the bend through the guide.

13. An endotracheal tube adaptor comprising:
a cap defining a guide and a holder, the cap is configured to couple to a connector of an endotracheal tube;
a pull component; and
a adjustment member having a first end, a second end, a first portion extending from the first end, and a second portion extending from the second end, and
wherein the first end of the adjustment member is coupled to the holder, the second portion of the adjustment member extends through the guide, and the second end of the adjustment member is coupled to the pull component.

14. The endotracheal tube adaptor of claim 13, wherein the adjustment member includes a bend extending between the first portion and the second portion.

15. The endotracheal tube adaptor of claim 13, wherein the guide is positioned radially outward from the holder.

16. The endotracheal tube adaptor of claim 13, wherein the holder is located at a central axis of the cap.

17. A method comprising:
inserting an endotracheal tube into a mouth or nose of a subject, the endotracheal tube including a connector and a tube extending distally from the connector;
providing tension on a pull line extending through a guide located proximate the connector to apply a curve to the tube; and
advancing the tube with the curve over an epiglottis and into a trachea of the subject.

18. The method of claim 17, further comprising:
threading the pull line through the guide.

19. The method of claim 18, wherein a distal end of the pull line is coupled to the distal end of the tube.

20. The method of claim 17, wherein a distal end of the pull line is coupled to a distal end of a compression component coupled to the connector.
